(19) United States
(12) Patent Application Publication
(10) Pub. No.: US 2007/0134130 A1
(43) Pub. Date: Jun. 14, 2007

(54) Biological specimen collection and transfer device and method of use

(75) Inventors: Timothy Hutchins, Attleboro Falls, MA (US); Robert Sakal, Bolton, MA (US)

Correspondence Address: VISTA IP LAW GROUP LLP, 12930 Saratoga Avenue, Suite D-2, Saratoga, CA, 95070, US

(73) Assignee: Cytyc Corporation, Marlborough, MA (US)

(21) Appl. No.: 11/298,952

(22) Filed: Dec. 9, 2005

(51) Int. Cl. G01N 33/00; C12M 1/34; B01D 61/00

(52) U.S. Cl. 422/63; 210/767; 435/287.3

(57) ABSTRACT

A filtration based biological specimen collection and transfer system includes a vial cassette configured for accessibly storing a plurality of sample vials, a filter cassette configured for accessibly storing a plurality of filter cartridges, a slide cassette configured for accessibly storing a plurality of specimen slides, a specimen slide processor configured to receive a sample vial, a filter cartridge, and a specimen slide, a transport system including a movable arm having an effecter engagement mechanism; and a plurality of effecters. Each effecter is configured for engaging the movable arm, for retrieving the sample vials, the filter cartridges, or the specimen slides from the respective vial cassette, filter cassette, or slide cassette and positioning them in the specimen slide processor, and for retrieving the sample vials, the filter cartridges, or the specimen slides from the specimen slide processor.

### FIELD OF INVENTION

The present invention generally relates to devices and methods for preparing biological specimens, and more particularly, to filtration based devices and methods for preparing biological specimens.

### DESCRIPTION OF RELATED ART

Many medical diagnostic tests, such as pap smears, require a physician to collect cells by brushing and/or scraping a skin or mucous membrane in a target area with an instrument. The collected cells are typically smeared (“fixed”) onto a slide, and stained to facilitate examination under a microscope by a cytotechnologist and/or pathologist. For example, a pathologist may employ a polychrome technique, characterized by staining the nuclear part of the cells, to determine the presence of dysplasia or neoplasia. The pathologist may also apply a counter-stain for viewing the cytoplasm of the cells. Because the sample may contain debris, blood, mucus and other obscuring artifacts, the test may be difficult to evaluate, and may not provide an accurate diagnostic assessment of the collected sample.

Cytology based on the collection of the exfoliated cells into a liquid preservative offers many advantages over the traditional method of smearing the cells directly onto the slide. A slide can be prepared from the cell suspension using a filter transfer technique, as disclosed in U.S. Pat. Nos. 6,572,824, 6,318,190, 5,772,818, 5,364,597 and 5,143,627, which are expressly incorporated herein by reference.

Filter transfer methods generally start with a collection of cells suspended in a liquid. These cells may be collected and dispersed into a liquid preservative or they may naturally exist in a collected biological liquid. Dispersion in liquid preservatives containing methanol, such as PreservCyt™ solution, breaks up mucus and lyses red blood cells and inflammatory cells, without affecting the cells of interest. The liquid is passed through a filter with a fixed diameter aperture covered by a membrane to concentrate and collect the cells. Debris, such as lysed blood cells and dispersed mucus, which flow through the pores of the membrane, are not collected on the membrane and are greatly reduced in the collected specimen by the combined methods of dispersion and filtering. Then the cells collected on the membrane are transferred onto a slide for further processing, such as visual examination.

Existing filter transfer methods include the steps of manually moving sample vials, filter cartridges and specimen slides to a specimen slide processor. These methods also include manually removing caps from and reattaching caps to sample vials, manually reading the indicia on the sample vials, manually labeling slides with indicia corresponding to that on the sample vials, and manually recording the correlation between samples and slides. These tasks are repetitious and time consuming. In addition to taking up valuable laboratory technician time, manually processing large numbers of samples can potentially lead to errors because of human involvement in the handling of samples vials and specimen slides.

Alternative filter transfer methods include the use of automated devices, such as elevators and graspers, which perform some of the above-described manual steps. The cumulative size of these automated devices requires that automated filtration based biological specimen preparation devices be large.

### SUMMARY OF THE INVENTION

In one embodiment, a filtration based biological specimen collection and transfer system is provided, the system including a vial cassette configured for accessibly storing a plurality of sample vials; a filter cassette configured for accessibly storing a plurality of filter cartridges; a slide cassette configured for accessibly storing a plurality of specimen slides; a specimen slide processor configured to receive a sample vial, a filter cartridge, and a specimen slide; a transport system including a movable arm having an effecter engagement mechanism; and a plurality of effecters. Each effecter is configured for engaging the movable arm, for retrieving the sample vials, the filter cartridges, or the specimen slides from the respective vial cassette, filter cassette, or slide cassette and positioning them in the specimen slide processor, and for retrieving the sample vials, the filter cartridges, or the specimen slides from the specimen slide processor.

The system may further include a vial handler configured for retrieving a sample vial from the vial cassette and positioning it in the specimen slide processor, and for retrieving the sample vial from the specimen slide processor and repositioning it in the vial cassette. The vial handler may further include a cap manipulator configured to remove a vial cap from and reattach the vial cap to the sample vial.

The plurality of effecters may include a filter handler configured for retrieving a filter cartridge from the filter cassette and positioning it in the specimen slide processor, and for retrieving the filter cartridge from the specimen slide processor. The filter handler may further include an elongated member configured to pierce a membrane in the filter cartridge.

The plurality of effecters may also further include a slide handler configured for retrieving a specimen slide from the slide cassette and positioning it in the specimen slide processor, and for retrieving the specimen slide from the specimen slide processor. By way of non-limiting example, the slide handler may include a dispenser configured to apply a fluid onto the specimen slide. By way of non-limiting examples, the dispenser can include a brush and/or a sprayer, and the fluid may include a stain and a fixative.

The system may further include a scanner configured to read vial indicia on the sample vial and/or a printer configured to mark the specimen slide with slide indicia.

Other and further embodiments, aspects and features of the invention will become apparent upon reviewing the following detailed description and accompanying figures.

### BRIEF DESCRIPTION OF THE DRAWINGS

In order to better understand and appreciate the invention, reference should be made to the drawings and accompany detailed description, which illustrate and describe exemplary embodiments thereof. For ease in illustration and understanding, similar elements in the different illustrated embodiments are referred to by common reference numerals. In particular:

FIG. 1 is a top schematic view of an exemplary biological specimen collection and transfer device according to one embodiment of the invention;

FIG. 2 is a perspective view of an exemplary sample vial configured for use with an biological specimen collection and transfer device according to one embodiment of the invention;

FIG. 3 is a side schematic view of an exemplary biological specimen collection and transfer device according to one embodiment of the invention;

FIG. 4 is a schematic view of an exemplary vial handler configured for use with an biological specimen collection and transfer device according to one embodiment of the invention;

FIG. 5 is a schematic view of an exemplary filter handler configured for use with an biological specimen collection and transfer device according to one embodiment of the invention; and

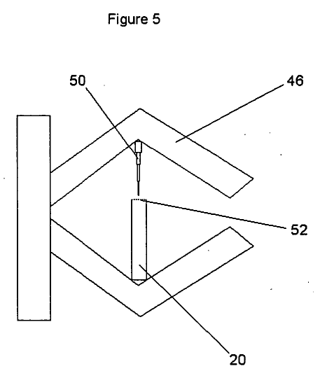

FIG. 6 is a schematic view of an exemplary slide handler configured for use with an biological specimen collection and transfer device according to one embodiment of the invention.

### DETAILED DESCRIPTION OF THE ILLUSTRATED EMBODIMENTS

In the following description of the illustrated embodiments, it will be understood by those skilled in the art that the drawings and specific components thereof are not necessarily to scale, and that various structural changes may be made without departing from the scope or nature of the various embodiments.

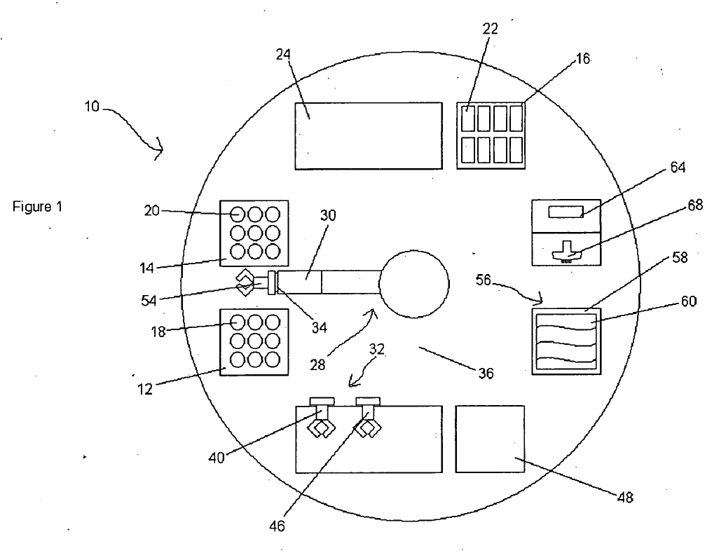

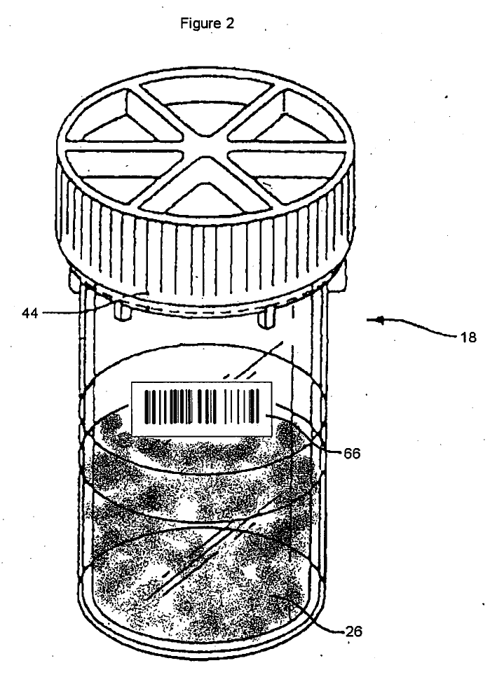

Referring to FIG. 1, a biological specimen collection and transfer device 10 is shown. The biological specimen collection and transfer device 10 includes a vial cassette 12, a filter cassette 14 and a slide cassette 16, which are configured for accessibly storing respective pluralities of sample vials 18, filter cartridges 20 and specimen slides 22. The collection and transfer device 10 also includes a specimen slide processor 24, which is configured to receive the sample vials 18, filter cartridges 20 and specimen slides 22. The specimen slide processor 24 is also configured to use a vacuum source (not shown) and the filter cartridge 20 to collect biological specimens 26 (FIG. 2) from the sample vial 18 and transfer it to the specimen slide 22.

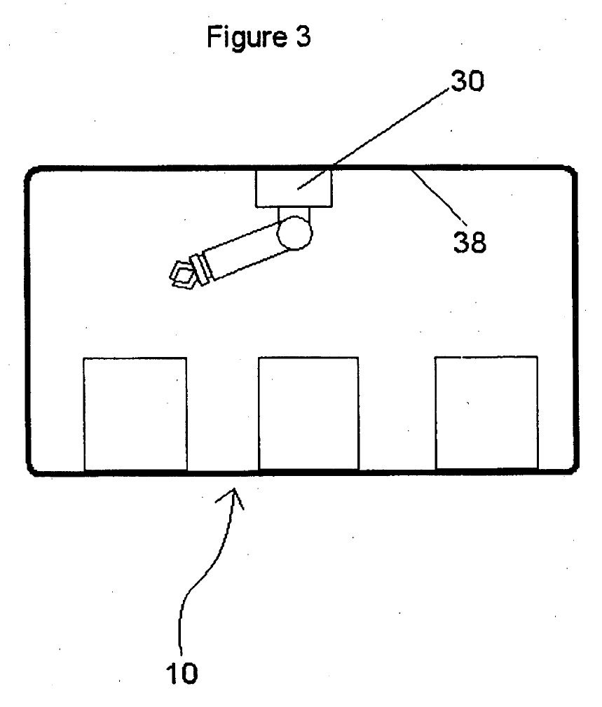

Further, the collection and transfer device 10 has a transport system 28, which includes a movable arm 30 and a plurality of effecters 32. The movable arm 30 has an effecter engagement mechanism 34, configured to reversibly connect to each of the plurality of effecters 32. A suitable movable arm 30 is the MELFA All-purpose Industrial Robot available from Mitsubishi Electric Automation, Inc. of Vernon Hills, Ill. The movable arm 30 may be mounted on a bottom surface 36 of the collection and transfer device 10. Alternatively, the movable arm 30 may be mounted to a top surface 38 of the collection and transfer device 10, as shown in FIG. 3.

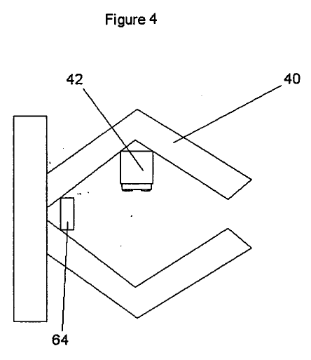

The effecters 32 include a vial hander 40, which is configured to retrieve the sample vials 18 from the vial cassette 12 and position them in the slide processor 24 and to retrieve the sample vials 18 from the slide processor 24 and reposition them in the vial cassette 12. The vial hander 40 also includes a cap manipulator 42 (FIG. 4) configured to remove vial caps 44 from and reattach vial caps 44 to the sample vials 18.

The effecters 32 also include a filter hander 46, which is configured to retrieve the filter cartridges 20 from the filter cassette 14 and position them in the slide processor 24 and to retrieve the filter cartridges 20 from the slide processor 24 and deposit them in a waste container 48. The filter hander 46 also includes a retractable spike 50 configured to pierce a membrane 52 in the filter cartridge 20 after use of the filter cartridge 20 and before the used filter cartridge is deposited in the waste container 48.

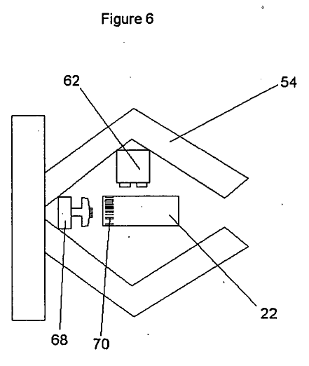

In addition, the effecters 32 include a slide handler 54, which is configured to retrieve the specimen slides 22 from the slide cassette 16 and position them in the slide processor 24 and to retrieve the specimen slides 22 from the slide processor 24 and position them in a slide unloading area 56. The unloading area 56 may be a bath 58 containing a fluid 60. The fluid 60 can be a stain or a fixative. Alternatively, as shown in FIG. 6, the slide handler 54 may include a dispenser 62 configured to apply the fluid 60 to the specimen slide 22. The dispenser 62 may either be a brush or a sprayer.

The collection and transfer device 10 also includes a scanner 64 configured to read vial indicia 66 on the sample vials 18. As shown in FIG. 4, the scanner 64 may be integrated into the vial hander 40. Moreover, the collection and transfer device 10 includes a printer 68 configured to mark the specimen slide 22 with slide indicia 70. As shown in FIG. 6, the printer 68 may be integrated into the slide handler 54.

In operation, the movable arm 30 reversibly connects to the vial hander 40 then the movable arm 30 and the vial hander 40 retrieve the sample vial 18 from the vial cassette 12. The integrated scanner 64 reads the vial indicia 66 from the sample vial 18. Then the movable arm 30 and the vial hander 40 position the sample vial 18 in the specimen slide processor 24.

The movable arm 30 disconnects from the vial hander 40 and connects to the filter hander 46. Then the movable arm 30 and the filter hander 46 retrieve the filter cartridge 20 from the filter cassette 14 and positions it in the specimen slide processor 24. The specimen slide processor 24 then collects the biological specimens 26 from the sample vial 18 onto the membrane 52 on the filter cartridge 20.

Meanwhile, the movable arm 30 disconnects from the filter hander 46 and connects to the slide handler 54. The movable arm 30 and the slide handler 54 then retrieve the specimen slide 22 from the slide cassette 16 and the integrated printer marks the specimen slide 22 with slide indicia 70. The movable arm and the slide handler 54 then position the specimen slide 22 in the specimen slide processor 24. The specimen slide processor 24 then transfers the collected biological specimens 26 from the membrane 52 onto the specimen slide 22.

The movable arm 30 and the slide handler 54 then retrieve the specimen slide 22 from the specimen slide processor 24 and positions it in the bath 58 containing a stain or/and a fixative. The movable arm 30 disconnects from the slide handler 54 and connects to the filter hander 46. The movable arm 30 and filter hander 46 retrieve the used filter cartridge 20 from the specimen slide processor 24. Then the attached spike 50 pierces the membrane 52 in the used filter cartridge 20, which is then deposited in the waste container 48.

The movable arm 30 disconnects from the filter hander 46 and connects to the vial hander 40. The movable arm 30 and vial hander 40 retrieve the sample vial 18 from the specimen slide processor 24 and repositions it in the vial cassette 12.

Although the collection and transfer device 10 and method are described for sequential batch processing, the collection and transfer device 10 and method can be reconfigured for parallel batch processing.

Although various embodiments of the invention have been shown and described herein, it should be understood that the above description and figures are for purposes of illustration only, and are not intended to be limiting of the invention, which is defined only by the appended claims and their equivalents.

What is claimed:

1. A filtration based biological specimen collection and transfer system, comprising:
a vial cassette configured for accessibly storing a plurality of sample vials;
a filter cassette configured for accessibly storing a plurality of filter cartridges;
a slide cassette configured for accessibly storing a plurality of specimen slides;
a specimen slide processor configured to receive a sample vial, a filter cartridge, and a specimen slide;
a transport system including a movable arm having an effecter engagement mechanism; and
a plurality of effecters, each effecter configured for engaging the movable arm, for retrieving the sample vials, the filter cartridges, or the specimen slides from the respective vial cassette, filter cassette, or slide cassette and positioning them in the specimen slide processor, and for retrieving the sample vials, the filter cartridges, or the specimen slides from the specimen slide processor.

2. The filtration based biological specimen collection and transfer system of claim 1, the plurality of effecters further comprising a vial handler configured for retrieving a sample vial from the vial cassette and positioning it in the specimen slide processor, and for retrieving the sample vial from the specimen slide processor and repositioning it in the vial cassette.

3. The filtration based biological specimen collection and transfer system of claim 2, the vial handler further comprising a cap manipulator configured to remove a vial cap from and reattach the vial cap to the sample vial.

4. The filtration based biological specimen collection and transfer system of claim 1, the plurality of effecters further comprising a filter handler configured for retrieving a filter cartridge from the filter cassette and positioning it in the specimen slide processor, and for retrieving the filter cartridge from the specimen slide processor.

5. The filtration based biological specimen collection and transfer system of claim 4, the filter handler further comprising an elongated member configured to pierce a membrane in the filter cartridge.

6. The filtration based biological specimen collection and transfer system of claim 1, the plurality of effecters further comprising a slide handler configured for retrieving a specimen slide from the slide cassette and positioning it in the specimen slide processor, and for retrieving the specimen slide from the specimen slide processor.

7. The filtration based biological specimen collection and transfer system of claim 6, the slide handler further comprising a dispenser configured to apply a fluid onto the specimen slide.

8. The filtration based biological specimen collection and transfer system of claim 1, further comprising a dispenser configured to apply a fluid onto the specimen slide.

9. The filtration based biological specimen collection and transfer system of claim 8, wherein the dispenser is selected from the group consisting of a brush and a sprayer.

10. The filtration based biological specimen collection and transfer system of claim 8, wherein the fluid is selected from the group consisting of a stain and a fixative.

11. The filtration based biological specimen collection and transfer system of claim 1, further comprising a scanner configured to read vial indicia on the sample vial.

12. The filtration based biological specimen collection and transfer system of claim 1, further comprising a printer configured to mark the specimen slide with slide indicia.

13. A filtration based biological specimen collection and transfer system, comprising:
a vial cassette configured for accessibly storing a plurality of sample vials;
a filter cassette configured for accessibly storing a plurality of filter cartridges;
a slide cassette configured for accessibly storing a plurality of specimen slides;
a specimen slide processor configured to receive a sample vial, a filter cartridge, and a specimen slide;
a transport system including a movable arm having an effecter engagement mechanism;
a vial handler configured for retrieving a sample vial from the vial cassette and positioning it in the specimen slide processor, and for retrieving the sample vial from the specimen slide processor and repositioning it in the vial cassette;
a filter handler configured for retrieving a filter cartridge from the filter cassette and positioning it in the specimen slide processor, and for retrieving the filter cartridge from the specimen slide processor; and
a slide handler configured for retrieving a specimen slide from the slide cassette and positioning it in the specimen slide processor, and for retrieving the specimen slide from the specimen slide processor.

14. A method of automatically collecting and transferring a biological specimen, comprising:
providing a movable arm having an effecter engagement mechanism;
engaging a vial handler with the movable arm;
positioning a sample vial with the vial handler in a specimen slide processor;
engaging a filter handler with the movable arm;
positioning a filter cartridge with the filter handler in the specimen slide processor;
engaging a slide handler with the movable arm; and
positioning a specimen slide with the slide handler in the specimen slide processor.

15. The method of claim 14, further comprising removing a cap from the sample vial and reattaching the cap onto the sample vial with the vial handler.

16. The method of claim 14, further comprising piercing a membrane in the filter cartridge with the filter handler.

17. The method of claim 14, further comprising
positioning the specimen slide in a dispenser; and
applying a stain to the specimen slide.

18. The method of claim 14, further comprising
positioning the specimen slide in a dispenser; and
applying a fixative to the specimen slide.

19. The method of claim 14, further comprising
positioning the sample vial proximate to a scanner; and
reading vial indicia on the sample vial.

20. The method of claim 14, further comprising
positioning the specimen slide in a printer; and
marking slide indicia on the specimen slide.
